(19) United States
(12) Patent Application Publication
(10) Pub. No.: US 2015/0057366 A1
(43) Pub. Date: Feb. 26, 2015

(54) SYNTHESIS OF ELEMICIN AND TOPICAL ANALGESIC COMPOSITIONS

(71) Applicant: Martin James LA GRANGE, Grafton. Auckland (NZ)

(72) Inventor: Martin James La Grange, Auckland (NZ)

(21) Appl. No.: 14/383,355

(22) PCT Filed: Mar. 5, 2013
(86) PCT No.: PCT/NZ2013/000030

§ 371 Date: Sep. 5, 2014

(30) Foreign Application Priority Data
Mar. 5, 2012 (NZ) 598560

(51) Int. Cl. C07C 41/16; C07C 45/27; C07C 41/26; A61K 31/09

(52) U.S. Cl. CPC C07C 41/16; A61K 31/09; C07C 45/27; C07C 41/26
USPC 514/720; 568/654

(57) ABSTRACT

The present invention is directed to the synthesis of elemicin and its isomeric form, and their use in topical analgesic formulations, including for dental and veterinary use. Various compositions include gels, films, dressings, cavity filling gels, having analgesic and/or antifungal properties.

### FIELD OF THE INVENTION

The present invention is directed to methods for the synthesis of elemicin, the use of elemicin in analgesia, as well as analgesic compositions based on same primarily directed to topical and dental use in humans and animals.

### BACKGROUND DESCRIPTION

By definition, analgesics reduce or eliminate pain. Analgesics financially represent a significant proportion of the world's pharmaceutical and veterinarian markets, and are very important to these industries.

While many analgesics are known, research continues into new products, but not only because of the financial importance of this market. Clinical testing occasionally discovers that prolonged use of certain products create unwanted side effects. Consequently many analgesics are unable to be taken by some patients, or can cause interactions with existing conditions or other medications; a well known example is the gastrointestinal effects of orally administered NSAIDs (non-steroidal anti-inflammatory drugs) a.k.a. NSAIAs.

While the present invention is directed primarily to topical and dental analgesia, similar issues still present themselves. Dermal site reactions can (statistically) potentially occur in some subjects with almost any composition, and additional complications exist if the topical composition is applied to open wounds or in dental use. For instance, one report indicates that topical application of NSAIDs (a significant topical analgesic) can result in adverse effects such as rashes and pruritis in approximately 15% of subjects [Drugs. 2000 September; 60(3):555-74 “Oral versus topical NSAIDs in rheumatic diseases: a comparison.” Heyneman C A, Lawless-Liday C, Wall G C.]

Amino-amide type anaesthetics, such as lidocaine (aka lignocaine) are commonly used in topical OTC (over the counter) formulations for topical dermal use (e.g. for sunburn) and dental use. However even then they are precluded in some situations due to some also being antiarrhythmic drugs.

Stronger analgesic compounds (opioids, cannibinoids, arylcyclohexylamines) are typically under tight regulatory controls and thus their use in OTC preparations are generally precluded due to the potential for abuse.

Many topical analgesics are also irritants and rubifacients (examples include compositions based on salicylates, capsaicin, etc.) or vasodilators (e.g. histamines and nicotinates). They are susceptible to producing unwanted site reactions, and typically are limited to intact dermal areas (not wounds) for short term use only.

Accordingly, there is a real need for new and additional topical analgesics, and especially those which may be suitable for OTC preparations.

Toxicity is a significant issue for any medicament. Even topically, considerations for any analgesic include local concentrations arising from transport through the dermis; whether the body possesses elimination mechanisms for any active component; toxicity when applied to open wounds; where the dermis is not intact; or to mucus and oral membranes, etc). Consideration also needs to be given to contraindications and side effects through repeated use. Not all available topical analgesics are always suitable in all situations, and thus there is a need for analgesics which extend the arsenal of available analgesics—particularly if it is suitable for use on open wounds and tissue, and/or mucal and oral membranes.

Accordingly, the market welcomes new alternative analgesic options as not all available options are suitable in every instance they may be required. Accordingly there is a real need for new analgesic formulations, and particularly if they potentially have relatively low side-effects in a significant population of users.

The present invention, as will be discussed later, uses a compound which occurs naturally, albeit in concentrations and forms in which any analgesic properties are negligible and have not been observed. The fact that one of the forms of this compound, elemicin, occurs naturally is of potential significance to the pharmaceutical industry. Many consumers are becoming wary of what they perceive as synthetic pharmaceuticals and prefer options based on naturally occurring substances—even if the active components in a formulation are manufactured synthetically. According, the present invention also potentially addresses another real and significant need in the industry—analgesic formulations based on naturally occurring substances.

Accordingly there is a need to provide an alternative topical analgesic composition or product.

At the very least it is an object of the present invention to provide the public with a useful alternative choice.

Aspects of the present invention will be described by way of example only and with reference to the ensuing description.

### GENERAL DESCRIPTION OF THE INVENTION

According to one aspect of the present invention there is provided a topical analgesic comprising at least 20% of at least one member of the group comprising elemicin and its isomers.

According to another aspect of the present invention there is provided a topical analgesic, substantially as described above, in which the composition comprises at least 40% of at least one member of the group comprising elemicin and its isomers.

According to another aspect of the present invention there is provided a topical analgesic, substantially as described above, in which there is both elemicin and iso-elemicin present in the composition.

According to another aspect of the present invention there is provided a topical analgesic, substantially as described above, in which the ratio of elemicin:iso-elemicin falls within the inclusive range of 800:200 to 998:2

According to another aspect of the present invention there is provided a topical analgesic, substantially as described above, in which the topical analgesic composition comprises a fluid composition.

According to another aspect of the present invention there is provided a topical analgesic, substantially as described above, in which the fluid composition is aqueous in nature.

According to another aspect of the present invention there is provided a topical analgesic, substantially as described above, which includes a humectant.

According to another aspect of the present invention there is provided a topical analgesic, substantially as described above, in which said humectant includes at least one member from the group comprising: polyethylene glycols with an average molecular weight less than 600, polypropylene glycols, block copolymers of polypropylene glycols, glycerol, sorbitol, xylitol, triacetin, sucrose octaacetate, and polyesters of sugars.

According to another aspect of the present invention there is provided a topical analgesic, substantially as described above, in which the composition comprises a gel in consistency.

According to another aspect of the present invention there is provided a topical analgesic, substantially as described above, which includes an aerogel.

According to another aspect of the present invention there is provided a topical analgesic, substantially as described above, in which the fluid composition is oil based in nature.

According to another aspect of the present invention there is provided a topical analgesic, substantially as described above, in which an oil based composition includes an oil which is able to pass through at least the epidermis.

According to another aspect of the present invention there is provided a topical analgesic, substantially as described above, which includes an oil extracted from plant material.

According to another aspect of the present invention there is provided a topical analgesic, substantially as described above, in which the plant oil is extracted from a component of at least one member of the following group: almonds, apricot kernels, avocados, baobab seeds, castor bean, coconut, evening primrose, grape seed, hazelnut, hemp seed, jojoba, kukui nut, macadamia nut, marula nut, nutmeg, mace, palm seed, peach kernels, pomegranate seed, pumpkin seed, olives, rosehips, safflower, sesame seeds, soy beans, sunflower seeds, calophyllum tacamahaca nuts, walnuts, and wheatgerm.

According to another aspect of the present invention there is provided a topical analgesic, substantially as described above, in which the fluid composition comprises a multiphase liquid composition.

According to another aspect of the present invention there is provided a topical analgesic, substantially as described above, in which the multiphase liquid composition comprises an aqueous phase, and an oil-based phase.

According to another aspect of the present invention there is provided a topical analgesic, substantially as described above, in which the topical analgesic comprises a non-fluid composition.

According to another aspect of the present invention there is provided a topical analgesic, substantially as described above, in which the topical analgesic forms a non-fluid composition once applied to a substrate.

According to another aspect of the present invention there is provided a topical analgesic, substantially as described above, in which the non-fluid composition comprises a flexible gel layer.

According to another aspect of the present invention there is provided a topical analgesic, substantially as described above, which includes at lease one component which is a member of the group comprising: carrageenan, aerogels, alginic acid, alginates, gelatines, xanthan gums, vegetable gums, agars, silicone gels, collagen, and pectins.

According to another aspect of the present invention there is provided a topical analgesic, substantially as described above, in which the non-fluid composition comprises a thin film.

According to another aspect of the present invention there is provided a topical analgesic, substantially as described above, in which the thin film is self-adhering to skin.

According to another aspect of the present invention there is provided a topical analgesic, substantially as described above, formulated for one or more of the following applications: for scar reduction, as a dressing for open wounds, as a dressing for sutured wounds, as a burn dressing, as a dressing for pruritis, and as an oral dressing.

According to another aspect of the present invention there is provided a topical analgesic, substantially as described above, substantially in the form of a powder.

According to another aspect of the present invention there is provided a topical analgesic, substantially as described above, in which the powdered composition comprises a hygroscopic and/or deliquescent component which will absorb moisture and form a gel or film like layer where applied.

According to another aspect of the present invention there is provided a topical analgesic, substantially as described above, in which the hygroscopic and/or deliquescent comprises at least one member of the group comprising: humectants, powdered aerogel agents.

According to another aspect of the present invention there is provided a topical analgesic, substantially as described above, in which there is present at least one member of the group comprising: carrageenan, alginic acid, alginates, gelatines, xanthan gums, vegetable gums, agars, silicone gels, collagen, and pectins.

According to another aspect of the present invention there is provided a topical analgesic, substantially as described above, in which the powdered composition is aerosol applied.

According to another aspect of the present invention there is provided a topical analgesic, substantially as described above, in an aerosol composition comprising a propellant in combination with at least crystalline elemicin

According to another aspect of the present invention there is provided a topical analgesic, substantially as described above, in which, when deposited onto a surface and any propellant or volatile components have evaporated, the residue contains at least 20% elemicin

According to another aspect of the present invention there is provided a topical analgesic, substantially as described above, in which the residue includes components which form a protective skin on the surface.

According to another aspect of the present invention there is provided a topical analgesic, substantially as described above, in which the residue includes one or members of the group comprising: collagen, carrageenan, alginic acid, alginates, gelatines, xanthan gums, vegetable gums, agars, silicone gels, and pectins.

According to another aspect of the present invention there is provided a topical analgesic, substantially as described above, in which the protective skin is permeable to oxygen.

According to another aspect of the present invention there is provided a topical analgesic, substantially as described above, in which the protective skin is permeable to water vapour

According to another aspect of the present invention there is provided a topical analgesic, substantially as described above, in which the permeability to water vapour is such that if applied to any open wound, it can still maintain a moist environment under the skin.

According to another aspect of the present invention there is provided a topical analgesic, substantially as described above, in which any and moisture permeability is similar to that of the human epidermis.

According to another aspect of the present invention there is provided a topical analgesic, substantially as described above, in which the skin is removable by washing with any one or more of: water, saline solution, ethanol, an ethanol solution in water comprising at least 20% ethanol.

According to a further aspect of the present invention there is provided a dressing with a contact area impregnated with a topical analgesic, substantially as described above.

According to another aspect of the present invention there is provided a dressing, substantially as described above, incorporating a topical analgesic substantially as described above.

According to another aspect of the present invention there is provided a dressing, substantially as described above, with a contact area impregnated with a topical analgesic substantially as described above and in which the analgesic composition comprises a dry analgesic composition.

According to yet a further aspect of the present invention there is provided a method for the synthesis of elemicin comprising steps of:

i) the conversion of eugenol to eugenol-5-aldehyde;
ii) the conversion of eugenol-5-aldehyde to 5-hydroxy-eugenol
iii) the conversion of 5-hydroxy-eugenol to elemicin.

According to another aspect of the present invention there is provided a method for the synthesis of elemicin, substantially as described above, in which the conversion of eugenol to eugenol-5-aldehyde comprises an ortho-oxidation reaction

According to another aspect of the present invention there is provided a method for the synthesis of elemicin, substantially as described above, in which the conversion of eugenol to eugenol-5-aldehyde comprises a step comprising the reaction of eugenol with hexamine under acidic conditions.

According to another aspect of the present invention there is provided a method for the synthesis of elemicin, substantially as described above, in which the acidic conditions for the conversion of eugenol to eugenol-5-aldehyde included the presence of glacial acetic acid.

According to another aspect of the present invention there is provided a method for the synthesis of elemicin, substantially as described above, in which the conversion of eugenol to eugenol-5-aldehyde comprises extraction and purification steps.

According to another aspect of the present invention there is provided a method for the synthesis of elemicin, substantially as described above, in which the conversion of eugenol-5-aldehyde to 5-hydroxy-eugenol comprises reaction with hydrogen peroxide in the presence of pyridine.

According to another aspect of the present invention there is provided a method for the synthesis of elemicin, substantially as described above, in which the conversion of 5-hydroxy-eugenol to elemicin comprises a methylation step.

According to another aspect of the present invention there is provided a method for the synthesis of elemicin, substantially as described above, in which the conversion of 5-hydroxy-eugenol to elemicin comprises the reaction of 5-hydroxy-eugenol with a methylation agent.

According to another aspect of the present invention there is provided a method for the synthesis of elemicin, substantially as described above, in which the conversion of 5-hydroxy-eugenol to elemicin comprises the reaction of 5-hydroxy-eugenol with dimethyl sulphate.

According to another aspect of the present invention there is provided a topical analgesic comprising elemicin synthesised according to a method substantially as described above.

According to another aspect of the present invention there is provided a topical analgesic, substantially as described above, in which there is at least 30% elemicin present.

According to another aspect of the present invention there is provided a topical analgesic, substantially as described above, in one or more of the forms in the group comprising: a dressing, a dermal covering, an oral ointment, an aerosol applied product, a gel, an oil based liquid, a multiphase liquid.

According to another aspect of the present invention there is provided a topical analgesic with synthesised elemicin, substantially as described above, for dental, veterinary, or pharmaceutical use.

According to yet an even further aspect of the present invention there is provided elemicin, as produced by a method substantially as described herein.

According to yet an even further aspect of the present invention there is provided the use of elemicin, as produced by a method substantially as described herein, in the preparation of a topical veterinary, dental, or pharmaceutical product.

According to yet an even further aspect of the present invention there is provided the use of a composition or product containing elemicin for analgesia in humans, other than as a method of medical treatment.

According to yet an even further aspect of the present invention there is provided the use of a composition or product containing elemicin for analgesia in non-human animals.

According to yet an even further aspect of the present invention there is provided the use of elemicin in the preparation of a topical analgesic.

Elemicin (1,2,3-Trimethoxy-5-prop-2-enylbenzene) is chemical compound with the structure

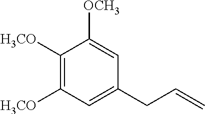

It is also known in the isomeric form iso-elemicin

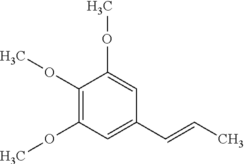

Elemicin is a known natural compound, occurring in nutmeg, mace (the aril of the nutmeg seed), and manila elemi (extracted from the resin of Canarium luzonicum). In the oils of nutmeg and manila elemi it typically occurs at a maximum concentration of around 2.4-3.0%, while in mace and some elemi oil we may occasionally obtain up to around 10-14% max. While elemi oil is used topically, analgesia is not a reported effect and work by the applicant suggests that analgesic effectiveness does not occur at such low doses—see below.

At the moment there is no major demand for elemicin (or iso-elemicin), partly due to its high cost arising from multiple extraction steps from expensive raw materials (nutmeg and mace oils) or via an uneconomical synthetic route such as proposed by Mauthner (detailed in the priority provisional specification). Furthermore, the prior art has failed to identify any valuable commercial use for elemicin—any properties attributed to nutmeg (Myristica fragrans) and mace are instead attributed to myristicin. In low doses nutmeg is reported as providing no noticeable physiological or neurological response. In higher doses psychoactive effects are reported—ascribed to the monoamine oxidase inhibitor, myristicin. Nutmeg poisoning from larger doses typically comprise convulsions, palpitations, nausea, dehydration, and generalised body pain. Accordingly the art tends to teach away from the use of components of nutmeg in analgesia.

Preliminary work by the applicant has indicated that compositions require at least 20% (and more preferably 30%) elemicin to start being effective as topical analgesics. More preferably higher concentrations will be required, though the choice of excipients, carriers, etc in a composition, and the nature of the composition/product, will have an effect on the optimal concentrations—hence some optimisation work is recommended for the skilled reader producing an idealised topical analgesic based on the disclosure of this document. Accordingly, effective analgesic compositions based on elemicin and/or iso-elemicin as the active component are absent in the prior art, and neither are they obvious given that the art has taught towards the use of other components, in natural products containing elemicin, for producing useful compositions.

Elemicin is approved in a number of regions for topical use, which represents a significant potential advantage as it avoids much of the testing and approvals required for compositions with new active compounds. This potentially represents a significant economic and commercial advantage, and has the potential to fulfil an ongoing longfelt want for economical alternative topical analgesics that can be realised relatively quickly into the market. It further exemplifies that the use of elemicin as an analgesic was not apparent, obvious, nor foreshadowed in the prior art and the industry.

The present invention involves two primary aspects—(i) a new economic synthetic route for the synthesis of elemicin, and (ii) topical analgesics based on elemicin.

### Synthesis of Elemicin

The present invention uses eugenol, a relatively cheap raw material, as its starting point. In the preferred embodiment there is a two stage ortho-oxidation process yielding 5-hydroxy-eugenol and a third stage in which this is converted to elemicin and myristicin derivatives.

A first stage of the reaction procedure converts the eugenol to eugenol-5-aldehyde. The preferred embodiment of the method reacts the eugenol with hexamine in the presence of acetic acid (preferably glacial). The reagents are further acidified, then neutralised with alkali (preferably sodium hydroxide) to produce an alkali metal salt of the aldehyde. This is ideally extracted and concentrated for the next step in the process.

In the preferred embodiment of a synthetic method, the eugenol-5-aldehyde is next converted to 5-hydroxy-eugenol. In the preferred embodiment, the eugenol-5-aldehyde is reacted with hydrogen peroxide in the presence of pyridine and alkali. This is later neutralised and the aldehyde product extracted for the final stage.

In the third stage of the preferred embodiment of a synthetic route, the hydroxy compound thus obtained is methylated with a methylating agent such as dimethyl sulphate. Optional isomerisation to iso-elemicin can be performed by alcoholic potassium hydroxide and subsequent oxidation with alkaline permanganate.

It should be realised, that for other embodiments, other reagents and reactants can be substituted into the various steps—this would be apparent to the nominal skilled worker and reader in light of the description given herein.

A more detailed description of a synthetic method will be given in the examples later herein.

### Topical Products and Compositions

The present invention in most of its embodiments is also intended and suitable for oral and dental use, and thus the term ‘topical’ as used herein is intended to include not only the dermis and epidermis, but any presenting exterior surface of the subject. This includes the mouth/oral cavity and nasal cavity. It also includes the exposed area of any wounds or areas to be treated.

Work thus far by the inventor also indicates that iso-elemicin is also effective in analgesic compositions and thus may be substituted for elemicin unless specifically stated otherwise. Hence, in the examples and descriptions given herein, we have referred primarily to elemicin for simplicity of description: it should be therefore taken into account that iso-elemicin may substitute some or all of the elemicin. However, the pharmacological activity of iso-elemicin compared to elemicin may vary in some individuals, and good practice indicates that any composition utilising iso-elemicin be optimised via trials for its particular intended purpose.

Preferred embodiments of the present invention typically comprise 20% (and ideally 30%) or more of elemicin (and/or iso-elemicin) though 40-60% is generally preferred for more effective analgesia. The lower percentages are typically preferred for antifungal analgesic compositions, in which the elemicin acts both as a mild analgesic and anti-fungal agent. In such cases the degree of analgesia need not always be as high (except in more severe cases where infection may also have occurred).

The use of elemicin (in both forms) as an antifungal agent does not appear to reported in the prior art either. Hence embodiments utilising this property are also considered within the general scope of the present invention. Other antifungal agents (e.g. ti tree oil [Melaleuca alternifolia], imidazoles, triazoles, thiazoles, neem seed oil, horopito extract [pseudowintera colorata]) may also be included in such compositions to enhance the antifungal properties of the composition.

Embodiments of the present invention may also take different forms. For simplicity of description we shall describe these generally as fluid and non-fluid compositions/products and it should be appreciated that there may be some overlap (e.g. thixotropic compositions, or film forming compositions, etc.)

In the fluid compositions, the product may typically comprise a liquid or gel. It may be thixotropic or non-thixotropic. It may be predominantly aqueous in nature, but may also be oil-based. Multiple phase compositions are also possible, typically being shaken before use.

It is noted that it is assumed that the reader is the nominal skilled reader familiar with standard techniques for producing topical compositions in the cosmetic and/or pharmaceutical/veterinary industries. Accordingly, standard techniques and compositions for producing cremes, gels, salves, ointments etc. shall not be described in detail but assumed to be part of the normal repertoire of knowledge and able to be used in conjunction with the present invention. The following parts of the specification draws upon, and builds upon, this knowledge.

Aqueous based compositions are typically based around various humectants and/or gelling agents (though various formulations are known in the relevant industries). Various examples include, but are not restricted to, one or members of the group comprising: collagen, carrageenan, alginic acid, alginates, gelatines, xanthan gums, vegetable gums, agars, silicone gels, and pectins. Aerogel products are another possibility, as well as other components commonly used in topical cosmetic and pharmaceutical compositions. These will become clearer from the examples given later herein, and will be able to be enabled by the skilled reader in light of the description given herein.

The typical aqueous compositions may take the form of a liquid or gel that can be applied or sprayed onto the desired area—the desired area in this case may include the skin, wounds, lesions, oral and nasal cavities. Typically they will be reapplied every several hours, depending on the precise embodiment, the area of application, and need of the subject.

They may be quite liquid (low viscosity) lotions and may dry on the area. They may also be of higher viscosity (e.g. gels, salves, and ointments) to provide more of a covering, and may be intended to be covered with a dressing.

Typical applications include for sunburn (such products may include other components such as aloe vera extracts, vitamin E, etc.), burns, abrasions, muscle sprains and injuries, arthritis, and other instances where a topical analgesic would be considered useful.

More advanced applications include wound and burn applications. In these types of cases a film forming product/composition or an existing film-type product might be used. Here the product can provide a protective film having analgesic properties. For wounds and burns this protective film can be quite important—maintaining a sterile environment, providing a moist environment to facilitate the body's own healing process, providing analgesia, etc. In such compositions it is also desirable to provide a readily removable film, enabling the film to be removed for further treatments, cleansing, inspection, debridement, etc. This has the potential to provide a readily removable protective film for burn and wound recovery and also having analgesic properties. There are significant different medical applications in this regard.

Aerogels in particular are suitable for this type of product, as are the various colloids etc. Here a suitable sterile film incorporating one of the forms of elemicin can be created, and cut to size for application to the wound, burn, or desired area. Most of these films will adhere slightly to moist areas so make it easier to apply and remain in place while a further optional dressing is applied. In some respects these products act as an artificial skin to protect the area. Depending on the specific gel/film forming formulation chosen the user can experiment to optimise a film idealised for a specific use—e.g. air and moisture permeabilities similar to the epidermis might be achieved, or a completely different set of values and parameters chosen. Here the user has a wide degree of choice.

It should also be considered that many aerogels (one example being those provided by the Evonik® corporation) also form gels with oils. Accordingly we can create a mixed aqueous/non-aqueous component film or gel which allows the user more control over the specific properties of the film or gel etc.

It is also considered that the product may be applied in a liquid form and will form a film once applied. This may be by evaporation of components (e.g. volatile components) though also may occur by mixing one or more components prior to use. For instance a colloid forming agent (suitable for increasing viscosity, and in this example may be a hydrocolloid) may be suspended in a carrier which does not allow it to form a colloid. This is then mixed with a second agent (e.g. water) prior to use and application. Possible colloids may include: carrageenan, alginic acid, alginates, gelatines, xanthan gums, vegetable gums, agars, silicone gels, collagen, cellulose products, starch products, and pectins.

One or two cross-linking agents with a cross-linking promoter may also be mixed and applied. An aerogel in powder form may be added to a suitable liquid portion to create a lotion or gel for application. As can be appreciated a variety of alternatives are available. Aglinates are one example of a cross-linkable polysaccharide, and is cross-linkable by Ca²⁺ ions.

The application method may be via a number of methods, including direct application, spray application, or the application of a dressing or film. Dressings may be conventional dressings with a contact area containing or impregnated with an elemicin (and/or isoelemicin) containing region—which can be an impregnated dressing area, or a layer of an elemicin (and/or isoelemicin) film.

Films may be adherent or attracted to the skin or wound areas to help them remain in place.

Semi-adherent silicone sheets are used over sutured areas to reduce scarring. Such sheets may be modified with a layer of a composition of the present invention to provide analgesia to the sutured area at the same time.

Aerogels, which are typically based on small silica particles (sometimes referred to as fumed or nano silicas) are a preferred component in many embodiments of the present invention. Depending upon the specific product, they are typically provided in a powdered form to be added to water and/or oils to produce a gel product—which can be gel-like, a viscous liquid, or a firm gel (suitable for films and protective coverings). They are generally suitable for, and approved for, most cosmetic and topical pharmaceutical uses and envisaged for use in various embodiments of the present invention.

Hence solid embodiments of the present invention include films, dressing, and covers (e.g. the modified silicone sheet examples). However it also includes powdered or granulated products which can be applied to an area and will then absorb or adsorb water from the environment (including the surface to which it is applied—e.g. a wound) to form a gel, film, or such like. Various humectants and/or aerogel products may be used in such embodiments. These compositions may be applied in a powdered or granular form to the desired area though aerosol compositions are also envisaged. These may activate once any volatile components/propellants have flashed off.

Oil based fluid compositions are also envisaged. In these embodiments they are predominantly oil based. They may also incorporate a variety of oils as carriers and components, including examples in which the plant oil is extracted from a component of at least one member of the following group: almonds, apricot kernels, avocados, baobab seeds, castor bean, coconut, evening primrose, grape seed, hazelnut, hemp seed, jojoba, kukui nut, macadamia nut, marula nut, nutmeg, mace, palm seed, peach kernels, pomegranate seed, pumpkin seed, olives, rosehips, safflower, sesame seeds, soy beans, sunflower seeds, calophyllum tacamahaca nuts, walnuts, and wheatgerm. Aerogels may also be combined with these components to alter the viscosity, and even produce non-fluid compositions and products. Other acceptable cosmetic and pharmaceutical components may also be used in various embodiments.

As can be appreciated there is a number of different embodiments of the present invention. These have the potential to take a number of different forms for topical pharmaceutical, dental, and pharmaceutical use. These will also be better exemplified with reference to the following examples, which are given by way of example only and not intended to be limiting.

### DESCRIPTION OF PREFERRED EMBODIMENT

### Example 1

### Synthesis of Elemicin

### Stage 1—Preparation of Eugenol-5-Aldehyde (X)

A solution of eugenol (10 cc) in glacial acetic acid (75 mL) was treated with hexamine (40 g). The mixture was heated with shaking over a wire gauze to get a clear solution (pale brown) and was kept in a boiling water-bath for six hours. The dark brown-red solution was treated while hot with a boiling mixture of concentrated hydrochloric acid (50 cc) and water (100 cc). Heating on the water-bath was continued for another five minutes and the mixture slowly cooled. It was extracted twice with ether and the ether extract washed with water. The clear ether solution was then shaken with 20% sodium hydroxide, added cautiously in small lots of 20 to 30 cc. On shaking, the lower aqueous layer was colourless showing that only acetic acid had been extracted. Two or three such extractions removed all the acetic acid. Further addition of the alkali solution gave a bright yellow crystalline solid. After shaking vigorously the layers were allowed to separate and the lower layer diluted further with 10% alkali in order to complete the precipitation of the yellow sodium salt of eugenol-5-aldehyde. It was filtered and washed with 10% alkali and ether. It was then dissolved in excess of water and filtered and the clear filtrate acidified. After leaving overnight the pale cream coloured solid that separated out was filtered, washed well with water and dried. Yield, 3 g.

The alkaline filtrate from the sodium salt was acidified and extracted with ether. The ether extract was washed with water, concentrated to a small bulk and shaken thoroughly with strong aqueous sodium bisulphite. The crystalline bisulphite addition compound was filtered and washed with ether. It was treated with dilute sulphuric acid and the mixture heated in a boiling water-bath till the solid disappeared giving oily drops. The mixture was cooled in the ice chest and the aldehyde that crystallised out was filtered. Yield, 1 g. The aldehyde samples thus obtained melted at 51-2°, when recrystallised from petroleum ether it was obtained in the form of pale yellow prisms melting at 53-54°. It was also purified by distillation under reduced pressure (20 mm), the fraction distilling at 148-50° being collected. On cooling in ice and scratching the sides of the tube, it rapidly solidified to give a mass of very pale yellow stout prisms melting at 53-4°. It was easily soluble in common organic solvents but sparingly in cold ligroin. In 10% sodium hydroxide it readily dissolved and the solution soon deposited yellow crystals of the sodium salt. With a drop of ferric chloride in alcoholic solution it gave a deep blue colour which did not change on further addition.

### Dinitrophenyl-Hydrazone of Eugenol-5-Aldehyde

An alcoholic solution of the aldehyde (0.2 g) was treated with dinitrophenyl-hydrazine (0.2 g) in alcohol (10 cc) and a few drops of concentrated hydrochloric acid. A copious crystalline precipitate of the dinitrophenyl-hydrazone was formed which was filtered after boiling for a few minutes. It was washed with alcohol and crystallised from ethyl acetate from which it separated as glistening orange red broad rectangular plates melting at 229-30°.

### Stage 2—Preparation of 5-Hydroxy-Eugenol (IX)

To a solution of eugenol-5-aldehyde (2.5 g) in pyridine (16 cc) was added one normal aqueous sodium hydroxide (19.5 cc) and the clear yellow solution treated dropwise with 6% hydrogen peroxide (9.6 cc) with vigorous shaking. The colour changed to red and when the solution developed turbidity a little more water was added in order to remove it. After leaving at the room temperature for 1.5 hours with occasional shaking, the solution was acidified with hydrochloric acid while cooling. The hydroxy compound separated as a heavy liquid; this separation was completed by adding common salt and the mixture was extracted thrice with ether. The ether extract was washed successively with hydrochloric acid, aqueous sodium bicarbonate and a small quantity of water. After drying over sodium sulphate it was evaporated to remove the solvent completely; a pale brown viscous oil was left behind (2.0 g). The product from three experiments was collected and distilled under reduced pressure (20 mm); the main fraction distilled at 176°. 5-hydroxy eugenol was a colourless viscous liquid. It was soluble in 5% aqueous sodium hydroxide and 10% aqueous sodium carbonate to give a deep brown solution. With a drop of ferric chloride in alcoholic solution it gave a deep violet colour changing to brownish violet and brown with another drop. After standing for a few minutes the colour changed to a stable olive green. It gave a yellow precipitate with lead acetate.

### Benzoylation: Dibenzoate of 5-Hydroxy Eugenol

The above dihydroxy compound was benzoylated by the Schotten-Baumann method. The benzoate separated from the alkaline solution in the form of a white sticky solid. It was extracted with ether, shaken well with 5% aqueous sodium hydroxide followed by water and dried over calcium chloride. On distilling off the ether a viscous liquid resulted which soon became an almost colourless crystalline solid. It was recrystallised from a mixture of ether and light petroleum from which it separated as colourless short rectangular prisms melting at 110-12°.

### Methylenation: Myristicin (II)

The dihydroxy compound (6 g) was dissolved in anhydrous acetone (200 cc), treated with methylene bromide (7.5 cc) and potassium carbonate (30 g) and refluxed for twenty hours. The solvent was then distilled off, the residue treated with water and the mixture extracted with ether. The ether extract was shaken with aqueous alkali, washed with water, dried over calcium chloride and distilled. The pale yellow oil left behind was distilled under reduced pressure (10 mm) when myristicin passed over at 138°. Yield, 2 g. Its refractive index was 1.5368 at 31° for white light, and 1.5343 at 30° and 1.5412 at 20° for the D line. When treated with a slight excess of bromine in petroleum ether solution it yielded dibromo-myristicin-dibromide which crystallised from acetone-methyl-alcohol mixture as colourless prismatic needles and melted at 128-29° alone or in admixture with a sample made from natural myristicin.

### Stage 3—Preparation of Elemicin (III)

The dihydroxy compound (6 g) was methylated in anhydrous acetone solution (100 cc) with dimethyl sulphate (9 cc) and potassium carbonate (20 g) by refluxing for six hours. The solvent was distilled off, the residue treated with water and the mixture extracted with ether. The ether extract was shaken with aqueous alkali, washed with water, dried over calcium chloride and distilled. The residual liquid was distilled under reduced pressure (10 mm) when elemicin passed over at 148-9°. It had a refractive index of 1.5245 at 31° for white light and 1.5240 at 30° and 1.5292 at 20° for the D line. Yield, 4 g. The synthetic sample of elemicin was subjected to isomerisation with alcoholic potash and subsequent oxidation with alkaline permanganate. The product was found to be trimethyl gallic acid, identical with an authentic sample.

### Summary

Employing the two stage process of ortho-oxidation eugenol is converted into 5-hydroxy eugenol.

Methylation of this yields elemicin and methylenation myristicin. This constitutes the most convenient synthesis, currently known to the inventor, of these naturally occurring compounds.

### Examples 2

In the following examples, percentages are by weight to a total of 100%

| Product | Variation | Ingredients | Comp |
|---|---|---|---|
| Analgesic Gel | Pure 001 | Elemicin | 30-70% |
| | | Evonik Aerogel 200 | To 100% |
| | | Pharma | |
| Analgesic Gel | Pure 002 | Elemicin | 70.00% |
| | | Evonik Aerogel 200 | 30.00% |
| | | Pharma | |
| Analgesic Gel | Pure 003 | Elemicin | 30.00% |
| | | Water | 20.00% |
| | | Evonik Aerogel 200 | 50.00% |
| | | Pharma | |
| Analgesic Gel | Pure 004 | Elemicin | 43.00% |
| | | Water | 15.50% |
| | | Mineral Oil | 5.00% |
| | | Evonik Aerogel 200 | 36.50% |
| | | Pharma | |
| Analgesic Cream | White 001 | Elemicin | 30.00% |
| | | Bees wax | 14.00% |
| | | Cetyl alcohol | 2.00% |
| | | Stearyl alcohol | 2.00% |
| | | Mineral oil | 23.00% |
| | | Lecithin | 2.00% |
| | | Borax | 1.00% |
| | | Water | 26.00% |
| | | Perfume | 0.02% |
| Analgesic Cream | White 002 | Elemicin | 40.00% |
| | | Bees wax | 14.00% |
| | | Cetyl alcohol | 2.00% |
| | | Stearyl alcohol | 2.00% |
| | | Mineral oil | 13.00% |
| | | Lecithin | 2.00% |
| | | Borax | 1.00% |
| | | Water | 26.00% |
| | | Perfume | 0.02% |
| Analgesic Cream | White 003 | Elemicin | 30% |
| | | Cetyl alcohol | 4.00% |
| | | Stearyl alcohol | 2.00% |
| | | Mineral oil | 23.00% |
| | | Water | 20.00% |
| | | Olive Oil | 8.00% |
| | | Lanolin | 4.00% |
| | | Gryceryl monostearate | 1.00% |
| | | Sodium laural sulphate | 1.00% |
| | | Perfume | 0.02% |
| | | Methyl paraben | 0.18% |
| Analgesic Cream | White 004 | Elemicin | 20% |
| | | Cetyl alcohol | 4.00% |
| | | Stearyl alcohol | 2.00% |
| | | Mineral oil | 33.00% |
| | | Water | 20.00% |
| | | Olive Oil | 8.00% |
| | | Lanolin | 4.00% |
| | | Gryceryl monostearate | 1.00% |
| | | Sodium laural sulphate | 1.00% |
| | | Perfume | 0.02% |
| | | Methyl paraben | 0.18% |
| | | Propyl paraben | 0.02% |
| Analgesic Cream | White 005 | Elemicin | 30% |
| | | Bees wax | 4.00% |
| | | Mineral oil | 7.00% |
| | | Borax | 0.25% |
| | | Water | 13.00% |
| | | Olive Oil | 5.00% |
| | | Lanolin | 37.50% |
| | | Spermacitin | 1.00% |
| | | Perfume | 0.02% |
| | | Propyl paraben | 2.00% |
| Analgesic Cream | White 005 | Elemicin | 20% |
| | | Bees wax | 4.00% |
| | | Mineral oil | 7.00% |
| | | Borax | 0.25% |
| | | Water | 18.00% |
| | | Olive Oil | 10.00% |
| | | Lanolin | 37.50% |
| | | Spermacitin | 1.00% |
| | | Perfume | 0.02% |
| | | Propyl paraben | 2.00% |
| Anti-Fungal Gel | ET 01 | Elemicin | 15-30% |
| | | Tea Tree Oil | 10.00% |
| | | Eucalyptus Oil | 10.00% |
| | | Evonik Aerogel 200 | To 100% |
| | | Pharma | |
| Anti-Fungal Gel | ET 02 | Elemicin | 15.00% |
| | | Tea Tree Oil | 12.50% |
| | | Evonik Aerogel 200 | 72.50% |
| | | Pharma | |

These represent some topical embodiments of the present invention by way of example only and may require the addition of water or solvents to achieve the required consistency desired in the final product.

### Example 3

This are two part reagents for mixing prior to use:

A first liquid portion comprises (by weight)

| | |
|---|---|
| elemicin or isoelemicin | 20-60% |
| ethanol | 0-20% |
| aerogel (dry product) | 5-20% |
| water | to 100% |

These are blended to provide a fluid component of low to medium viscosity

The second portion comprises either aerogel dry product, or aerogel in a viscous gel form. This second portion is blended with the first liquid portion prior to use, with the quantity of the second portion chosen to achieve the desired consistency in the end product.

Such compositions may be used in a variety of applications including over wounds, film forming artificial skin type applications, ointments for under dressings, cavity filling wound dressings, etc.

### Example 4

An anti-fungal composition from example 2 which also includes one or more members of the anti-fungal group comprising: ti tree oil [Melaleuca alternifolia], imidazoles, triazoles, thiazoles, neem seed oil, horopito extract [pseudowintera colorata])

### Example 5

A dry powder comprising at least 30% elemicin or isoelemicin in combination with dry components from the group in turn comprising: aerogel powders, dried hydrocolloids, inert cosmetically and pharmaceutically acceptable dry powdered agents, and dry flow agents.

Optional components may include, in dry powdered form, one or more of the following: antibiotics, anti-fungal agents, antioxidants, vitamins.

Other inert or active dry components may be optionally added to the composition.

### Example 6

A composition of example 5 in aerosol form. This may include one or more of the following components: collagen, film forming agents (such as used in some sunburn sprays), alcohol, antiseptic agents, volatile carriers, etc.

These may be used for analgesia but also anti-fungal treatment.

### Example 7

A gel composition of example 2 or 3 as a cavity filling ointment for wounds. Ideally this is a gel which can be applied to a wound cavity prior to covering with a dressing. This may include collagen.

### Example 8

A wound dressing comprising a standard dressing with a contact area impregnated with a composition such as described in any of examples 2 or 3.

The dressing may comprise a silicone sheet such as used for application over sutured incisions to reduce scarring.

### Example 9

A flexible film of elemicin in combination with an aerogel, colloid, or hydrocolloid, able to be trimmed or peeled from a backing for application to a desired surface.

Such films may be present on a dressing (see example 8) as opposed to impregnation.

### Examples 10

| Product | % Elemicin % | Purity Carrying Medium | Format |
|---|---|---|---|
| Wound Salve | 20% | 98% Clear Gel | Tube |
| Wound Salve | 30% | 98% Clear Gel | Tube |
| Wound Salve | 35% | 98% Clear Gel | Tube |
| Wound Salve | 40% | 98% Clear Gel | Tube |
| Wound Salve | 50% | 98% Clear Gel | Tube |
| Wound Salve | 65% | 98% Clear Gel | Tube |
| Wound Salve | 75% | 98% Clear Gel | Tube |
| Wound Salve | 20% | 98% White Gel | Tube |
| Wound Salve | 30% | 98% White Gel | Tube |
| Wound Salve | 35% | 98% White Gel | Tube |
| Wound Salve | 40% | 98% White Gel | Tube |
| Wound Salve | 50% | 98% White Gel | Tube |
| Wound Salve | 65% | 98% White Gel | Tube |
| Wound Salve | 75% | 98% White Gel | Tube |
| Wound Salve | 20% | 98% Coloured Gel | Tube |
| Wound Salve | 30% | 98% Coloured Gel | Tube |
| Wound Salve | 35% | 98% Coloured Gel | Tube |
| Wound Salve | 40% | 98% Coloured Gel | Tube |
| Anti fungal Gel | 20% | 98% Clear Gel | Tube |
| Anti Fungal Gel | 25% | 98% Clear Gel | Tube |
| Anti Fungal Spray | 20% | 98% Liquid | Spray |
| Anti Fungal Spray | 25% | 98% Liquid | Aerosol Spray |
| Anti Fungal Spray | 22.50% | 98% Powder | Aerosol Spray |
| Anti Fungal Spray | 25% | 98% Powder | Aerosol Spray |
| Anti Fungal Cream | 20% | 98% Cold Cream | Cream Pot |
| Anti Fungal Cream | 25% | 98% Cold Cream | Cream Pot |
| Anti Fungal Cream | 20% | 98% Cold Cream | Tube |
| Anti Fungal Cream | 25% | 98% Cold Cream | Tube |
| Antiseptic Gel | 30% | 98% Clear Gel | Tube |
| Antiseptic Gel | 35% | 98% Clear Gel | Tube |
| Antiseptic Spray | 30% | 98% Liquid | Spray |
| Antiseptic Spray | 35% | 98% Liquid | Aerosol Spray |
| Antiseptic Spray | 27.00% | 98% Powder | Aerosol Spray |
| Antiseptic Spray | 30% | 98% Powder | Aerosol Spray |
| Antiseptic Cream | 30% | 98% Cold Cream | Cream Pot |
| Antiseptic Cream | 35% | 98% Cold Cream | Cream Pot |
| Antiseptic Cream | 30% | 98% Cold Cream | Tube |
| Antiseptic Cream | 35% | 98% Cold Cream | Tube |
| Wound Dressing | 20% | 98% Clear Gel | Self-Adhesive Plaster |
| Wound Dressing | 22.50% | 98% Clear Gel | Self-Adhesive Plaster |
| Wound Dressing | 40% | 98% Clear Gel | Self-Adhesive Plaster |
| Wound Dressing | 45% | 98% Clear Gel | Self-Adhesive Plaster |
| Wound Dressing | 47.50% | 98% Clear Gel | Self-Adhesive Plaster |
| Wound Dressing | 20% | 98% Powder | Self-Adhesive Plaster |
| Wound Dressing | 22.50% | 98% Powder | Self-Adhesive Plaster |
| Wound Dressing | 40% | 98% Powder | Self-Adhesive Plaster |
| Wound Dressing | 45% | 98% Powder | Self-Adhesive Plaster |
| Wound Dressing | 47.50% | 98% Powder | Self-Adhesive Plaster |
| Wound Salve | 50% | 98% Coloured Gel | Tube |
| Wound Salve | 65% | 98% Coloured Gel | Tube |
| Wound Salve | 75% | 98% Coloured Gel | Tube |
| Sensitive Toothpaste | 17.50% | 98% Toothpaste | Toothpaste Tube |
| Sensitive Toothpaste | 18.50% | 98% Toothpaste | Toothpaste Tube |
| Dental Cream | 18.50% | 98% Clear Gel | Tube |
| Dental Cream | 20% | 98% Clear Gel | Tube |
| Dental Cream | 35% | 98% Clear Gel | Tube |
| Dental Cream | 50% | 98% Clear Gel | Tube |
| Dental Cream | 75% | 98% Clear Gel | Tube |
| Sunburn Gel | 20% | 98% Clear Gel | Tube |
| Sunburn Gel | 30% | 98% Clear Gel | Tube |
| Sunburn Gel | 35% | 98% Clear Gel | Tube |
| Sunburn Gel | 40% | 98% Clear Gel | Tube |
| Sunburn Gel | 50% | 98% Clear Gel | Tube |
| Sunburn Gel | 65% | 98% Clear Gel | Tube |
| Sunburn Gel | 75% | 98% Clear Gel | Tube |
| Sunburn Cream | 20% | 98% White Gel | Tube |
| Sunburn Cream | 30% | 98% White Gel | Tube |
| Sunburn Cream | 35% | 98% White Gel | Tube |
| Sunburn Cream | 40% | 98% White Gel | Tube |
| Sunburn Cream | 50% | 98% White Gel | Tube |
| Sunburn Cream | 65% | 98% White Gel | Tube |
| Sunburn Cream | 75% | 98% White Gel | Tube |
| Sunburn Cream, Coloured | 20% | 98% Coloured Gel | Tube |
| Sunburn Cream, Coloured | 30% | 98% Coloured Gel | Tube |
| Sunburn Cream, Coloured | 35% | 98% Coloured Gel | Tube |
| Sunburn Cream, Coloured | 40% | 98% Coloured Gel | Tube |
| Sunburn Cream, Coloured | 50% | 98% Coloured Gel | Tube |
| Sunburn Cream, Coloured | 65% | 98% Coloured Gel | Tube |
| Sunburn Cream, Coloured | 75% | 98% Coloured Gel | Tube |
| Sunburn Gel Spray | 20% | 98% Clear Gel | Aerosol Spray |
| Sunburn Gel Spray | 30% | 98% Clear Gel | Aerosol Spray |
| Sunburn Gel Spray | 35% | 98% Clear Gel | Aerosol Spray |
| Sunburn Gel Spray | 40% | 98% Clear Gel | Aerosol Spray |
| Sunburn Gel Spray | 50% | 98% Clear Gel | Aerosol Spray |
| Sunburn Gel Spray | 65% | 98% Clear Gel | Aerosol Spray |
| Sunburn Gel Spray | 75% | 98% Clear Gel | Aerosol Spray |
| Sunburn Powder Spray | 20% | 98% Powder | Aerosol Spray |
| Sunburn Powder Spray | 30% | 98% Powder | Aerosol Spray |
| Sunburn Powder Spray | 35% | 98% Powder | Aerosol Spray |
| Sunburn Powder Spray | 40% | 98% Powder | Aerosol Spray |
| Sunburn Powder Spray | 50% | 98% Powder | Aerosol Spray |
| Sunburn Powder Spray | 65% | 98% Powder | Aerosol Spray |
| Sunburn Powder Spray | 75% | 98% Powder | Aerosol Spray |
| Anti fungal Gel | 20% | 85% Clear Gel | Tube |
| Anti Fungal Gel | 25% | 85% Clear Gel | Tube |
| Anti Fungal Spray | 20% | 85% Liquid | Spray |
| Anti Fungal Spray | 25% | 85% Liquid | Aerosol Spray |
| Anti Fungal Spray | 22.50% | 85% Powder | Aerosol Spray |
| Anti Fungal Spray | 25% | 85% Powder | Aerosol Spray |
| Anti Fungal Cream | 20% | 85% Cold Cream | Cream Pot |
| Anti Fungal Cream | 25% | 85% Cold Cream | Cream Pot |
| Anti Fungal Cream | 20% | 85% Cold Cream | Tube |
| Anti Fungal Cream | 25% | 85% Cold Cream | Tube |
| Antiseptic Gel | 30% | 85% Clear Gel | Tube |
| Antiseptic Gel | 35% | 85% Clear Gel | Tube |
| Antiseptic Spray | 30% | 85% Liquid | Spray |
| Antiseptic Spray | 35% | 85% Liquid | Aerosol Spray |
| Antiseptic Spray | 27.00% | 85% Powder | Aerosol Spray |
| Antiseptic Spray | 30% | 85% Powder | Aerosol Spray |
| Antiseptic Cream | 30% | 85% Cold Cream | Cream Pot |
| Antiseptic Cream | 35% | 85% Cold Cream | Cream Pot |
| Antiseptic Cream | 30% | 85% Cold Cream | Tube |
| Antiseptic Cream | 35% | 85% Cold Cream | Tube |
| Wound Salve | 20% | 75% Coloured Gel | Tube |
| Wound Salve | 30% | 75% Coloured Gel | Tube |
| Wound Salve | 35% | 75% Coloured Gel | Tube |
| Wound Salve | 40% | 75% Coloured Gel | Tube |
| Wound Salve | 50% | 75% Coloured Gel | Tube |
| Wound Salve | 65% | 75% Coloured Gel | Tube |
| Wound Salve | 75% | 75% Coloured Gel | Tube |
| Sensitive Toothpaste | 17.50% | 75% Toothpaste | Toothpaste Tube |
| Sensitive Toothpaste | 18.50% | 75% Toothpaste | Toothpaste Tube |
| Dental Cream | 18.50% | 75% Clear Gel | Tube |
| Dental Cream | 20% | 75% Clear Gel | Tube |
| Dental Cream | 35% | 75% Clear Gel | Tube |
| Dental Cream | 50% | 75% Clear Gel | Tube |
| Dental Cream | 75% | 75% Clear Gel | Tube |
| Sunburn Gel | 20% | 75% Clear Gel | Tube |
| Sunburn Gel | 30% | 75% Clear Gel | Tube |
| Sunburn Gel | 35% | 75% Clear Gel | Tube |
| Sunburn Gel | 40% | 75% Clear Gel | Tube |
| Sunburn Gel | 50% | 75% Clear Gel | Tube |
| Sunburn Gel | 65% | 75% Clear Gel | Tube |
| Sunburn Gel | 75% | 75% Clear Gel | Tube |
| Sunburn Cream | 20% | 75% White Gel | Tube |
| Sunburn Cream | 30% | 75% White Gel | Tube |
| Sunburn Cream | 35% | 75% White Gel | Tube |
| Sunburn Cream | 40% | 75% White Gel | Tube |
| Sunburn Cream | 50% | 75% White Gel | Tube |
| Sunburn Cream | 65% | 75% White Gel | Tube |
| Sunburn Cream | 75% | 75% White Gel | Tube |
| Sunburn Cream, Coloured | 20% | 75% Coloured Gel | Tube |
| Sunburn Cream, Coloured | 30% | 75% Coloured Gel | Tube |
| Sunburn Cream, Coloured | 35% | 75% Coloured Gel | Tube |
| Sunburn Cream, Coloured | 40% | 75% Coloured Gel | Tube |
| Sunburn Cream, Coloured | 50% | 75% Coloured Gel | Tube |
| Sunburn Cream, Coloured | 65% | 75% Coloured Gel | Tube |
| Sunburn Cream, Coloured | 75% | 75% Coloured Gel | Tube |
| Sunburn Gel Spray | 20% | 75% Clear Gel | Aerosol Spray |
| Sunburn Gel Spray | 30% | 75% Clear Gel | Aerosol Spray |
| Sunburn Gel Spray | 35% | 75% Clear Gel | Aerosol Spray |
| Sunburn Gel Spray | 40% | 75% Clear Gel | Aerosol Spray |
| Sunburn Gel Spray | 50% | 75% Clear Gel | Aerosol Spray |
| Sunburn Gel Spray | 65% | 75% Clear Gel | Aerosol Spray |
| Sunburn Gel Spray | 75% | 75% Clear Gel | Aerosol Spray |
| Sunburn Powder Spray | 20% | 75% Powder | Aerosol Spray |
| Sunburn Powder Spray | 30% | 75% Powder | Aerosol Spray |
| Sunburn Powder Spray | 35% | 75% Powder | Aerosol Spray |
| Sunburn Powder Spray | 40% | 75% Powder | Aerosol Spray |
| Sunburn Powder Spray | 50% | 75% Powder | Aerosol Spray |
| Sunburn Powder Spray | 65% | 75% Powder | Aerosol Spray |
| Sunburn Powder Spray | 75% | 75% Powder | Aerosol Spray |
| Wound Dressing | 20% | 85% Clear Gel | Self-Adhesive Plaster |
| Wound Dressing | 22.50% | 85% Clear Gel | Self-Adhesive Plaster |
| Wound Dressing | 40% | 85% Clear Gel | Self-Adhesive Plaster |
| Wound Dressing | 45% | 85% Clear Gel | Self-Adhesive Plaster |
| Wound Dressing | 47.50% | 85% Clear Gel | Self-Adhesive Plaster |
| Wound Dressing | 20% | 85% Powder | Self-Adhesive Plaster |
| Wound Dressing | 22.50% | 85% Powder | Self-Adhesive Plaster |
| Wound Dressing | 40% | 85% Powder | Self-Adhesive Plaster |
| Wound Dressing | 45% | 85% Powder | Self-Adhesive Plaster |
| Wound Dressing | 47.50% | 85% Powder | Self-Adhesive Plaster |
| Wound Salve | 20% | 85% Clear Gel | Tube |
| Wound Salve | 30% | 85% Clear Gel | Tube |
| Wound Salve | 35% | 85% Clear Gel | Tube |
| Wound Salve | 40% | 85% Clear Gel | Tube |
| Wound Salve | 50% | 85% Clear Gel | Tube |
| Wound Salve | 65% | 85% Clear Gel | Tube |
| Wound Salve | 75% | 85% Clear Gel | Tube |
| Wound Salve | 20% | 85% White Gel | Tube |
| Wound Salve | 30% | 85% White Gel | Tube |
| Wound Salve | 35% | 85% White Gel | Tube |
| Wound Salve | 40% | 85% White Gel | Tube |
| Wound Salve | 50% | 85% White Gel | Tube |
| Wound Salve | 65% | 85% White Gel | Tube |
| Wound Salve | 75% | 85% White Gel | Tube |
| Wound Salve | 20% | 85% Coloured Gel | Tube |
| Wound Salve | 30% | 85% Coloured Gel | Tube |
| Wound Salve | 35% | 85% Coloured Gel | Tube |
| Wound Salve | 40% | 85% Coloured Gel | Tube |
| Wound Salve | 50% | 85% Coloured Gel | Tube |
| Wound Salve | 65% | 85% Coloured Gel | Tube |
| Wound Salve | 75% | 85% Coloured Gel | Tube |
| Sensitive Toothpaste | 17.50% | 85% Toothpaste | Toothpaste Tube |
| Sensitive Toothpaste | 18.50% | 85% Toothpaste | Toothpaste Tube |
| Dental Cream | 18.50% | 85% Clear Gel | Tube |
| Dental Cream | 20% | 85% Clear Gel | Tube |
| Dental Cream | 35% | 85% Clear Gel | Tube |
| Dental Cream | 50% | 85% Clear Gel | Tube |
| Dental Cream | 75% | 85% Clear Gel | Tube |
| Sunburn Gel | 20% | 85% Clear Gel | Tube |
| Sunburn Gel | 30% | 85% Clear Gel | Tube |
| Sunburn Gel | 35% | 85% Clear Gel | Tube |
| Sunburn Gel | 40% | 85% Clear Gel | Tube |
| Sunburn Gel | 50% | 85% Clear Gel | Tube |
| Sunburn Gel | 65% | 85% Clear Gel | Tube |
| Sunburn Gel | 75% | 85% Clear Gel | Tube |
| Sunburn Cream | 20% | 85% White Gel | Tube |
| Sunburn Cream | 30% | 85% White Gel | Tube |
| Sunburn Cream | 35% | 85% White Gel | Tube |
| Sunburn Cream | 40% | 85% White Gel | Tube |
| Sunburn Cream | 50% | 85% White Gel | Tube |
| Sunburn Cream | 65% | 85% White Gel | Tube |
| Sunburn Cream | 75% | 85% White Gel | Tube |
| Sunburn Cream, Coloured | 20% | 85% Coloured Gel | Tube |
| Sunburn Cream, Coloured | 30% | 85% Coloured Gel | Tube |
| Sunburn Cream, Coloured | 35% | 85% Coloured Gel | Tube |
| Sunburn Cream, Coloured | 40% | 85% Coloured Gel | Tube |
| Sunburn Cream, Coloured | 50% | 85% Coloured Gel | Tube |
| Sunburn Cream, Coloured | 65% | 85% Coloured Gel | Tube |
| Sunburn Cream, Coloured | 75% | 85% Coloured Gel | Tube |
| Sunburn Gel Spray | 20% | 85% Clear Gel | Aerosol Spray |
| Sunburn Gel Spray | 30% | 85% Clear Gel | Aerosol Spray |
| Sunburn Gel Spray | 35% | 85% Clear Gel | Aerosol Spray |
| Sunburn Gel Spray | 40% | 85% Clear Gel | Aerosol Spray |
| Sunburn Gel Spray | 50% | 85% Clear Gel | Aerosol Spray |
| Sunburn Gel Spray | 65% | 85% Clear Gel | Aerosol Spray |
| Sunburn Gel Spray | 75% | 85% Clear Gel | Aerosol Spray |
| Sunburn Powder Spray | 20% | 85% Powder | Aerosol Spray |
| Sunburn Powder Spray | 30% | 85% Powder | Aerosol Spray |
| Sunburn Powder Spray | 35% | 85% Powder | Aerosol Spray |
| Sunburn Powder Spray | 40% | 85% Powder | Aerosol Spray |
| Sunburn Powder Spray | 50% | 85% Powder | Aerosol Spray |
| Sunburn Powder Spray | 65% | 85% Powder | Aerosol Spray |
| Sunburn Powder Spray | 75% | 85% Powder | Aerosol Spray |
| Anti fungal Gel | 20% | 75% Clear Gel | Tube |
| Anti Fungal Gel | 25% | 75% Clear Gel | Tube |
| Anti Fungal Spray | 20% | 75% Liquid | Spray |
| Anti Fungal Spray | 25% | 75% Liquid | Aerosol Spray |
| Anti Fungal Spray | 22.50% | 75% Powder | Aerosol Spray |
| Anti Fungal Spray | 25% | 75% Powder | Aerosol Spray |
| Anti Fungal Cream | 20% | 75% Cold Cream | Cream Pot |
| Anti Fungal Cream | 25% | 75% Cold Cream | Cream Pot |
| Anti Fungal Cream | 20% | 75% Cold Cream | Tube |
| Anti Fungal Cream | 25% | 75% Cold Cream | Tube |
| Antiseptic Gel | 30% | 75% Clear Gel | Tube |
| Antiseptic Gel | 35% | 75% Clear Gel | Tube |
| Antiseptic Spray | 30% | 75% Liquid | Spray |
| Antiseptic Spray | 35% | 75% Liquid | Aerosol Spray |
| Antiseptic Spray | 27.00% | 75% Powder | Aerosol Spray |
| Antiseptic Spray | 30% | 75% Powder | Aerosol Spray |
| Antiseptic Cream | 30% | 75% Cold Cream | Cream Pot |
| Antiseptic Cream | 35% | 75% Cold Cream | Cream Pot |
| Antiseptic Cream | 30% | 75% Cold Cream | Tube |
| Antiseptic Cream | 35% | 75% Cold Cream | Tube |
| Wound Dressing | 20% | 75% Clear Gel | Self-Adhesive Plaster |
| Wound Dressing | 22.50% | 75% Clear Gel | Self-Adhesive Plaster |
| Wound Dressing | 40% | 75% Clear Gel | Self-Adhesive Plaster |
| Wound Dressing | 45% | 75% Clear Gel | Self-Adhesive Plaster |
| Wound Dressing | 47.50% | 75% Clear Gel | Self-Adhesive Plaster |
| Wound Dressing | 20% | 75% Powder | Self-Adhesive Plaster |
| Wound Dressing | 22.50% | 75% Powder | Self-Adhesive Plaster |
| Wound Dressing | 40% | 75% Powder | Self-Adhesive Plaster |
| Wound Dressing | 45% | 75% Powder | Self-Adhesive Plaster |
| Wound Dressing | 47.50% | 75% Powder | Self-Adhesive Plaster |
| Wound Salve | 20% | 75% Clear Gel | Tube |
| Wound Salve | 30% | 75% Clear Gel | Tube |
| Wound Salve | 35% | 75% Clear Gel | Tube |
| Wound Salve | 40% | 75% Clear Gel | Tube |
| Wound Salve | 50% | 75% Clear Gel | Tube |
| Wound Salve | 65% | 75% Clear Gel | Tube |
| Wound Salve | 75% | 75% Clear Gel | Tube |
| Wound Salve | 20% | 75% White Gel | Tube |
| Wound Salve | 30% | 75% White Gel | Tube |
| Wound Salve | 35% | 75% White Gel | Tube |
| Wound Salve | 40% | 75% White Gel | Tube |
| Wound Salve | 50% | 75% White Gel | Tube |
| Wound Salve | 65% | 75% White Gel | Tube |
| Wound Salve | 75% | 75% White Gel | Tube |

### Example 11

A topical cosmetic composition, such as a moisturising agent, foundation, sun-cream, etc. which contains at least 25% elemicin.

It should be noted herein that aerogel is sometimes used in reference to the dry powder and other times to the resulting gel when mixed with a carrier fluid—it is envisaged that the skilled addressee will be aware of the intended usage. A preferred aerogel is a fumed silica product currently produced by Evonik Industries and currently marketed under their Aerosil® brand. A preferred product of theirs in the present invention is their Aerosil 200 Pharma which is described as a high purity amorphous anhydrous colloidal silicon dioxide for use in pharmaceutical products. Equivalent type products from other manufacturers can be used.

Aspects of the present invention have been described by way of example only and it should be appreciated that modifications and additions may be made thereto without departing from the spirit or scope of the present invention as described herein.

It should also be understood that the term “comprise” where used herein is not to be considered to be used in a limiting sense. Accordingly, ‘comprise’ does not represent nor define an exclusive set of items, but includes the possibility of other components and items being added to the list.

This specification is also based on the understanding of the inventor regarding the prior art. The prior art description should not be regarded as being authoritative disclosure on the true state of the prior art but rather as referencing considerations brought to the mind and attention of the inventor when developing this invention.

1-61. (canceled)

62. A topical analgesic comprising at least 20% of at least one member of the group comprising elemicin and its isomers.

63. A topical analgesic as claimed in claim 62 in which the composition comprises at least 40% of at least one member of the group comprising elemicin and its isomers.

64. A topical analgesic as claimed in claim 63 in which there is both elemicin and iso-elemicin present in the composition.

65. A topical analgesic as claimed in claim 64 in which the ratio of elemicin:iso-elemicin falls within the inclusive range of 800:200 to 998:2.

66. A topical analgesic as claimed in claim 65 which said humectant includes at least one member from the group comprising: polyethylene glycols with an average molecular weight less than 600, polypropylene glycols, block copolymers of polypropylene glycols, glycerol, sorbitol, xylitol, triacetin, sucrose octaacetate, and polyesters of sugars.

67. A topical analgesic as claimed in claim 66 in which the composition includes an oil extracted from plant material selected from at least one member of the group comprising almonds, apricot kernels, avocados, baobab seeds, castor bean, coconut, evening primrose, grape seed, hazelnut, hemp seed, jojoba, kukui nut, macadamia nut, marula nut, palm seed, peach kernels, pomegranate seed, pumpkin seed, olives, rosehips, safflower, sesame seeds, soy beans, sunflower seeds, calophyllum tacamahaca nuts, walnuts, and wheatgerm.

68. A topical analgesic as claimed in claim 67 which includes at least one component which is a member of the group comprising: carrageenan, aerogels, alginic acid, alginates, gelatines, xanthan gums, vegetable gums, agars, silicone gels, collagen, and pectins.

69. A topical analgesic as claimed in claim 68 in which there is present at least one member of the group comprising: carrageenan, alginic acid, alginates, gelatines, xanthan gums, vegetable gums, agars, silicone gels, collagen, and pectins.

70. A method for the synthesis of elemicin comprising steps of:
i) the conversion of eugenol to eugenol-5-aldehyde;
ii) the conversion of eugenol-5-aldehyde to 5-hydroxy-eugenol
iii) the conversion of 5-hydroxy-eugenol to elemicin.

71. A method for the synthesis of elemicin as claimed in claim 70 in which the conversion of eugenol to eugenol-5-aldehyde comprises an ortho-oxidation reaction.

72. A method for the synthesis of elemicin as claimed in claim 71 in which the conversion of eugenol to eugenol-5-aldehyde comprises a step comprising the reaction of eugenol with hexamine under acidic conditions.

73. A method for the synthesis of elemicin as claimed claim 72 in which the acidic conditions for the conversion of eugenol to eugenol-5-aldehyde included the presence of glacial acetic acid.

74. A method for the synthesis of elemicin as claimed in claim 73 in which the conversion of eugenol to eugenol-5-aldehyde comprises extraction and purification steps.

75. A method for the synthesis of elemicin as claimed in claim 74 in which the conversion of eugenol-5-aldehyde to 5-hydroxy-eugenol comprises reaction with hydrogen peroxide in the presence of pyridine.

76. A method for the synthesis of elemicin as claimed in claim 75 in which the conversion of 5-hydroxy-eugenol to elemicin comprises a methylation step.

77. A method for the synthesis of elemicin as claimed in claim 76 in which the conversion of 5-hydroxy-eugenol to elemicin comprises the reaction of 5-hydroxy-eugenol with a methylation agent.

78. A method for the synthesis of elemicin as claimed in claim 77 in which the conversion of 5-hydroxy-eugenol to elemicin comprises the reaction of 5-hydroxy-eugenol with dimethyl sulphate.
